SEQUENCE LISTING (3 sequences)

SEQ ID NO: 1 - protein, 243 aa, Severe acute respiratory syndrome-related coronavirus
  SARS-CoV-2 RBD protein
MFVFLVLLPLVSSQRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFHHHHHH

SEQ ID NO: 2 - protein, 26 aa, Severe acute respiratory syndrome-related coronavirus
  SARS-CoV-2 #1
VEGFNCYFPLQSYGFQPTNGVGYQPY

SEQ ID NO: 3 - protein, 30 aa, Severe acute respiratory syndrome-related coronavirus
  SARS-CoV-2 #2
KVGGNYNYLYRLFRKSNLKPFERDISTEIY

(19) United States
(12) Patent Application Publication
(10) Pub. No.: US 2026/0115276 A1
(43) Pub. Date: Apr. 30, 2026

(54) TREATMENT AND/OR PREVENTION OF AN INFECTION BY MONO/DIVALENT AND POLYVALENT ANTIGEN PARTICLE-MEDIATED IMMUNE RESPONSES

(71) Applicants: VACCINVENT GMBH, Freiburg im Breisgau (DE); UNIVERSITÄT ULM, Ulm (DE)

(72) Inventors: Hassan JUMAA-WEINACHT, Ulm (DE); Hend ABDELRASOUL, Ulm (DE); Corinna SETZ, Beimerstetten (DE)

(21) Appl. No.: 18/273,692

(22) PCT Filed: Jan. 28, 2022
(86) PCT No.: PCT/EP2022/052147

§ 371 Date: Jul. 21, 2023

(30) Foreign Application Priority Data
Jan. 28, 2021 (WO) PCT/EP2021/052000
Aug. 5, 2021 (EP) 21189997.6

(51) Int. Cl. A61K 39/215; A61K 39/00; A61K 39/39; A61P 37/04

(52) U.S. Cl. CPC A61K 39/215; A61K 39/39; A61P 37/04; A61K 2039/52; A61K 2039/6006; A61K 2039/6037; A61K 2039/6081; A61K 2039/627

(57) ABSTRACT

The invention pertains to means and methods for the targeted modulation of immune responses by bringing into contact a B-cell with mono/divalent antigen particles and/or polyvalent antigen particles. The targeted modulation of B-cell immunity can be used in the therapy of infections. The invention is predicated on the observation that the combination of polyvalent antigenic structures and mono/divalent antigenic structures harbour the ability to potentiate immune responses against antigens.

### RELATED APPLICATIONS

The instant application is a 35 U.S.C. § 371 filing of International Patent Application No. PCT/EP2022/052147, filed Jan. 28, 2022, which claims priority to European Patent Application No. 21189997.6, filed Aug. 5, 2021, and International Patent Application No. PCT/EP2021/052000, filed Jan. 28, 2021, the entire contents of which are incorporated herein by reference for all purposes.

### SEQUENCE LISTING

The instant application contains a Sequence Listing which has been submitted electronically in TXT format and is hereby incorporated by reference in its entirety. Said ASCII file, created on Mar. 4, 2024, is named 745019_VOS9-013US_ST25.txt and is 3,310 bytes in size.

The invention pertains to means and methods for the targeted modulation of immune responses by bringing into contact a B-cell with mono/divalent antigen particles and/or polyvalent antigen particles. The targeted modulation of B-cell immunity can be used in the therapy of infections. The invention is predicated on the observation that the combination of polyvalent antigenic structures and mono/divalent antigenic structures harbour the ability to potentiate immune responses against antigens.

The vaccine is one of the greatest inventions of modern medicine and is the most economic and effective weapon for resisting pathogens such as viruses and virus-induced diseases for human beings. Because of the use of vaccines, humans have successfully eradicated smallpox, essentially eradicated polio, and successfully controlled most diseases that once afflict humans, such as tuberculosis, measles, diphtheria, tetanus, and the like.

Currently, the development of vaccines relies on the traditional model of B cell selection and development proposing that central tolerance mechanisms remove autoreactive B cell specificities resulting in a peripheral B cell repertoire devoid of autoreactive potential.

Since the outbreak of the current global COVID-19 pandemic, laboratories of various countries intensified the work on new ways to induce immune responses and improve vaccines.

Hence, there is a continued need for novel and flexible approaches for a controllable modulation of immune responses in order to improve prevention and/or treatment against pathogens.

The above technical problem is solved by the embodiments disclosed herein and as defined in the claims.

Accordingly, the invention relates to, inter alia, the following embodiments:
1. A composition, comprising:
   (i) a mono/divalent antigen particle, comprising an antigenic portion comprising one or two antigenic structures capable of inducing an antibody-mediated immune response against a target antigen, wherein the target antigen is a pathogen-associated antigen, and
   (ii) a polyvalent antigen particle comprising an antigenic portion comprising more than two antigenic structures capable of inducing an antibody-mediated immune response against the target antigen and wherein the more than two antigenic structures are cross-linked, wherein the target antigen is a pathogen-associated antigen;
   for use in the treatment and/or prevention of an infection.
2. The composition for use of embodiment 1, wherein the more than two antigenic structure comprise multiple identical antigenic structures.
3. The composition for use of embodiment 1 or 2, wherein the polyvalent antigen particle further comprises a carrier portion which is coupled to the antigenic portion and/or wherein the mono/divalent antigen particle further comprises a carrier portion which is coupled to the antigenic portion.
4. The composition for use of embodiment 3, wherein the carrier portion comprises a structure selected from the group of polypeptides, immune CpG islands, limpet hemocyanin (KLH), tetanus toxoid (TT), cholera toxin subunit B (CTB), bacteria or bacterial ghosts, liposome, chitosome, virosomes, microspheres, dendritic cells, particles, microparticles, nanoparticles, or beads.
5. The composition for use of embodiment 1 to 4, wherein the cross-link in the polyvalent-antigen particle is a chemical cross-link, such as a bis-maleimide mediated cross-link, or is a protein cross-link, such as a biotin-streptavidin mediated cross-link.
6. The composition for use of embodiments 1 to 5, wherein the polyvalent-antigen particle comprises a complex of the following formula A-L-A, wherein A is a target antigen comprising portion, and wherein L is the linker of the cross link, preferably wherein L is a bismaleimide, and most preferably the complex is of the following structure (I), wherein R is a target antigen comprising portion:

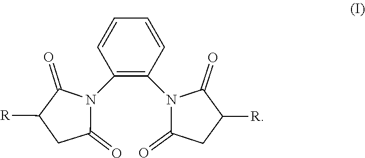

7. The composition for use of embodiments 1 to 5, wherein the polyvalent-antigen particle comprises a linker with a crosslink reactive group for protein conjugation, preferably a linker with a crosslink reactive group for stable protein conjugation.
8. The composition for use of embodiment 7, wherein the crosslink reactive group is a group selected from carboxyl-to-amine reactive groups, amine-reactive groups, sulfhydryl-reactive groups, aldehyde-reactive groups and photoreactive groups.
9. The composition for use of embodiment 7, wherein the crosslink reactive group is a group selected from carbodiimide, NHS ester, imidoester, pentafluorophenyl ester, hydroxymethyl phosphine, maleimide, haloacetyl, hydrazide, alkoxyamine, diazirine and aryl azide.
10. The composition for use of embodiments 1 to 9, wherein the polyvalent antigen particle comprises the at least two copies of the antigenic structure in spatial proximity to each other, preferably within a range of 3 nm to 20 nm.
11. The composition for use of embodiments 1 to 10, wherein the pathogen-associated antigen comprises at least one agent selected from the group of nucleic acid, carbohydrate and peptide.
12. The composition for use of embodiments 1 to 11, wherein the polyvalent antigen particle is linked to an adjuvant, preferably wherein the polyvalent particle is covalently linked to an adjuvant.
13. The composition for use of embodiment 12, wherein the adjuvant is IgG.
14. The composition for use of embodiments 1 to 13, wherein treatment and/or prevention comprises at least two administration time points.
15. The composition for use of embodiment 14, wherein prevention comprises administering the mono/divalent antigen particle before the polyvalent antigen particle.
16. The composition for use of embodiments 14 to 15, wherein the treatment and/or prevention comprises at least two administration time points for the mono/divalent antigen particle and least two administration time points for the polyvalent antigen particle.
17. The composition for use of any of the previous embodiments wherein the antibody-mediated immune response is an IgG and/or IgM mediated immune response.
18. The composition for use of embodiments 1 to 17, wherein the pathogen is at least one pathogen selected from the group of parasite, bacterium and virus.
19. The composition for use of embodiments 1 to 18, wherein the infection is a viral infection.
20. The composition for use of embodiment 19, wherein the viral infection is a coronavirus infection.
21. The composition for use of embodiment 20, wherein the coronavirus infection is a SARS-COV-2 infection.
22. The composition for use of embodiment 21, wherein the pathogen-associated antigen comprises an amino acid sequence derived from the corona virus spike protein, such as a receptor binding domain (RBD) sequence, preferably the complete RBD sequence, or a sequence comprising at least 80% sequence identity to the amino acid sequence of the SARS-COV-2 RBD amino acid sequence (SEQ ID NO: 1).
23. A method for producing an antibody that binds to a pathogen-associated antigen comprising the steps of:
   (1) administration of:
   (i) a mono/divalent particle, comprising an antigenic portion comprising one or two antigenic structures capable of inducing an antibody-mediated immune response against a target antigen, wherein the target antigen comprises a pathogen-associated antigen, and
   (ii) a polyvalent antigen particle comprising an antigenic portion comprising more than two antigenic structures capable of inducing an antibody-mediated immune response against the target antigen and wherein the more than two antigenic structures are cross-linked, wherein the target antigen comprises a pathogen-associated antigen,
   to a subject and/or a cell capable of producing antibodies; and
   (2) isolating an antibody from the subject and/or cell, wherein the antibody binds to the target antigen.
24. A polynucleotide encoding:
   (i) a mono/divalent antigen particle, comprising an antigenic portion comprising one or two antigenic structures capable of inducing an antibody-mediated immune response against a target antigen, wherein the target antigen comprises a pathogen-associated antigen, and
   (ii) a polyvalent antigen particle comprising an antigenic portion comprising more than two antigenic structures capable of inducing an antibody-mediated immune response against the target antigen and wherein the more than two antigenic structures are cross-linked, wherein the target antigen comprises a pathogen-associated antigen;
   for use in the treatment and/or prevention of an infection.
25. A vector comprising the polynucleotide for use of embodiment 24.

Accordingly, the invention relates to a composition, comprising: (i) a mono/divalent antigen particle, comprising an antigenic portion comprising one or two antigenic structures capable of inducing an antibody-mediated immune response against a target antigen, wherein the target antigen comprises a pathogen-associated antigen, and (ii) a polyvalent antigen particle comprising an antigenic portion comprising more than two antigenic structures capable of inducing an antibody-mediated immune response against the target antigen and wherein the more than two antigenic structures are cross-linked, wherein the target antigen comprises a pathogen-associated antigen; for use in the treatment and/or prevention of an infection.

The term “valent” as used within the current application denotes the presence of a specified number of binding sites in an antibody or antigen, respectively, molecule. As such a binding site of an antibody is a paratope, whereas a binding site in the antigen is generally referred to as an epitope. A natural antibody for example or a full-length antibody according to the invention has two binding sites and is bivalent. Antigen proteins are mono/divalent (when present as monomers), however, if such antigen proteins are provided as multimers they may comprise more than one identical epitope and therefore are polyvalent, which may be bivalent, trivalent, tetravalent, etc. As such, the terms “trivalent”, denote the presence of three binding sites in an antibody molecule. As such, the terms “tetravalent”, denote the presence of four binding sites in an antibody molecule.

The term “mono/divalent antigen particle”, as described herein, refers to a molecule or molecule-complex, such as a protein, or protein complexes, which are antigenic, and therefore capable of stimulating an immune response in a vertebrate. Typically, a mono/divalent antigen particle is composed of (i) one antigenic portion comprising not more than two of an antigenic structure capable of inducing an antibody mediated immune response against such antigenic structure or (ii) two antigenic portions comprising not more than one of an antigenic structure capable of inducing an antibody mediated immune response against such antigenic structure. The term “mono/divalent antigen particle”, as used herein refers to a monovalent antigen particle, a divalent particle or a combination of a monovalent antigen particle and a divalent antigen particle. In some embodiments, the term “mono/divalent antigen particle” described herein additionally includes a polyvalent precursor that degrades into mono/divalent antigen particle in the body of a subject prior to eliciting a substantial immune response (e.g. a prodrug that is activated upon contact with enzymes of the body).

The term “antigenic structure”, as used herein, refers to fragment of an antigenic agent (e.g. protein) that retains the capacity of stimulating an antibody mediated immune response. Such an antigenic structure is understood to provide the antigenic determinant or “epitope” which refers to the region of a molecule that specifically reacts with an antibody, more specifically that reacts with a paratope of an antibody. In preferred embodiments of the invention a mono/divalent antigen particle of the invention comprises not more than two copies of one specific epitope of the antigenic structure. Hence, preferably only one/two antibody molecules of a certain antibody species having a specific paratope may bind to a mono/divalent antigen particle according to the invention.

The term “polyvalent antigen particle” shall in the context of the herein disclosed invention refer to a molecule or molecule-complex, such as a protein, or protein complexes, which are antigenic, and therefore capable of stimulating an immune response in a vertebrate. In the invention, unlike mono/divalent antigen particles, a polyvalent antigen particle is composed of an antigenic portion comprising more than two antigenic structures capable of inducing an antibody-mediated immune response. In some embodiments, the term “polyvalent antigen particle” described herein additionally includes a lower-valent (e.g. mono/divalent) precursor that forms the polyvalent antigen particle in the body of a subject prior to eliciting a substantial immune response (e.g. an agent that is complexed and/or polymerized upon contact with enzymes of the body).

The term “treatment” (and grammatical variations thereof such as “treat” or “treating”), as used herein, refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, decreasing the rate of disease progression, amelioration or palliation of the disease state, and remission or improved prognosis.

The term “prevention”, as used herein, relates to the capacity to prevent, minimize or hinder the onset or development of a disorder, disease or condition before its onset.

In some embodiments, the disease or disorder described herein refers to one or more symptoms and/or complications of the disease or disorder.

In preferred embodiments of the invention, a polyvalent antigen particle of the invention comprises more than two copies of one specific epitope of the antigenic structure. In some embodiments, the polyvalent antigen particle of the invention comprises more than three copies of one specific epitope of the antigenic structure.

Hence, preferably more than one antibody molecule of a certain antibody species having a specific paratope may bind to a mono/divalent antigen particle according to the invention. Such polyvalent antigen particles may have a structure that the more than one antigenic structures are covalently or non-covalently cross-linked with each other. Preferably, the more than one antigenic structure comprised in an antigenic portion of the polyvalent antigen particle comprises multiple identical antigenic structures.

In context of the invention the mono/divalent antigen particle of the invention is often referred to as “soluble” particle or antigen whereas the polyvalent antigen particle is referred to as “complexed” particle or antigen.

The term “target antigen”, as used herein, refers to any molecule or structure that comprises an antigenic structure. A target antigen of the invention can be a natural and/or synthetic immunogenic substance, such as a complete, fragment or portion of an immunogenic substance, and wherein the immunogenic substance may be selected from a nucleic acid, a carbohydrate, a peptide, or any combination thereof.

The term “cross-link”, as used herein, refers to a bond that links at least two antigenic structures with each other, wherein the cross-linked complex has different physical properties than the separated antigenic structures. In some embodiments, the cross-linked complex is less soluble than the separated antigenic structures. In some embodiments, the cross-link described herein comprises at least one covalent bond. In some embodiments, the cross-link described herein comprises at least one ionic bond.

The term “pathogen”, as used herein, refers to an agent that may cause a disease, such as an infectious disease, in a subject. Pathogens include, for example, bacteria, viruses, prions, fungi, protozoans, helminths, nematodes, and any other pathogenic agent which may sicken a subject or, if transmitted from a subject who may not suffer disease, could cause disease in a further subject to which the pathogen is transmitted.

The term “pathogen-associated antigen”, as used herein, refers to any antigenic molecule, structure or agent that can be found in a pathogen, preferably to a molecule, structure or agent that is specific for the pathogen (e.g. pathogen-specific nucleic acid, carbohydrate, peptide and/or protein). Therefore, the pathogen-associated antigen is preferably a structure that is found in the pathogen but not or not substantially in the body of a subject or has a higher biological relevance in the pathogen than in the body of the subject. In some embodiments the pathogen-associated antigen described herein is a carbohydrate and/or peptide that is found on the surface of the pathogen. In some embodiments the pathogen-associated antigen described herein is a carbohydrate and/or peptide required for the entrance of the pathogen into a cell.

The term “infection”, as used herein, refers to the invasion and multiplication of a pathogen in the body of a subject.

In context of the present invention, it is distinguished between mono/divalent antigen particles opposed to polyvalent antigen particles. Each particle is considered as a single molecular entity, which may comprise covalently or non-covalently connected portions. However, according to the present invention each particle has an immunogenic activity towards a certain antigen. The mono/divalent antigen particle is therefore understood to comprise only one or two antigenic structure that is/are able to elicit an immune response to the antigen whereas the polyvalent antigen particle comprises three or more, four or more copies of such antigenic structures. In context of the present invention sometimes also the terms “soluble” antigen is used for the mono/divalent antigen particle opposed to “complex” antigen for the polyvalent antigen particle. It is understood that in most instances the antigenic structure comprises or consists of an epitope that elicits an antibody immune response, and in turn is a binding site for an antibody produced upon a cell-mediated immune response. In other words, the invention distinguishes between a presentation of immune eliciting epitopes as soluble single epitope or in a complexed array identical epitope.

The present invention is predicated at least in part upon the surprising finding that antigens may induce different immune responses depending on whether they are presented to immune cells as soluble antigens or as polyvalent antigen particles. The combination of soluble antigens and complexed polyvalent antigen particle can increase the immune, reduces the need and/or improves the effect of adjuvants and/or reduces the required dose (see e.g. FIG. 1). Furthermore, the combination described herein can suppress the production of protective IgM antibodies (FIG. 2-8).

These findings establish a dynamic model of B cell activation, in which immune responses are regulated by relative amounts of antigen forms B cells thereby allowing an unrestricted potential of adaptive immune responses.

Therefore, means and methods described herein provide a novel and versatile way to induce and alter an immune response. The antigen(s) presented on the antigen particle can be efficiently adapted to newly emerging pathogens, pathogen mutations and/or resistance mechanisms. The production of the antigen particles can be standardized and do not have the biological variation of other immune response inducers such as attenuated or inactivated virus vaccines. Furthermore, the distribution of the antigen particles described herein can be controlled and predicted unlike other immune response inducers such as mRNA vaccines.

Accordingly, the invention is at least in part based on the surprising finding that a combination of mono/divalent and polyvalent antigen particles can be used to potentiate and/or sustain antibody production.

In certain embodiments, the invention relates to the composition for use of the invention, wherein the more than two antigenic structure comprise multiple identical antigenic structures.

Hence, preferably more than one antibody molecule of a certain antibody species having a specific paratope may bind to a polyvalent antigen particle according to the invention. Such polyvalent antigen particles may have a structure that the more than one of an antigenic structures are covalently or non-covalently cross-linked with each other. A polyvalent antigen particle, therefore, in preferred embodiments comprises complex comprising at least two identical epitopes and therefore, which allow for a binding of two antibodies to the polyvalent antigen particle at the same time. Preferably, the more than one of an antigenic structure comprised in an antigenic portion of the polyvalent antigen particle comprises multiple identical antigenic structures. A polyvalent antigen particle therefore, in preferred embodiments comprises complex comprising at least two, at least three or at least four identical epitopes, which allow for a binding of two antibodies to the polyvalent antigen particle at the same time.

The composition comprising such particles according to the invention can modulate an immune response (see e.g. FIGS. 1-8).

Accordingly, the invention is at least in part based on the surprising finding that a plurality of linked identical structures can modulate the immune response to a target antigen as described herein.

In certain embodiments, the invention relates to the composition for use of the invention, wherein the polyvalent antigen particle further comprises a carrier portion which is coupled to an antigenic portion and/or wherein the mono/divalent antigen particle further comprises a carrier portion which is coupled to an antigenic portion.

The term “carrier portion” in context of the herein disclosed invention preferably relates to a substance or structure that presents or comprises the antigenic structures of the particles of the invention.

In certain embodiments, the invention relates to composition for use of the invention, wherein the carrier portion comprises a structure selected from the group of polypeptides, immune CpG islands, limpet hemocyanin (KLH), tetanus toxoid (TT), cholera toxin subunit B (CTB), bacteria or bacterial ghosts, liposome, chitosome, virosomes, microspheres, dendritic cells, particles, microparticles, nanoparticles, or beads.

In some embodiments of the invention, the polyvalent-antigen particle further comprises a carrier portion which is coupled to an antigenic portion, optionally via a linker, and wherein the carrier, and optionally the linker, does not comprise another copy of the antigenic structure, and wherein the carrier portion, and optionally the linker, is not capable of eliciting a antibody-mediated immune response against the target antigen. In another alternative or additional embodiment of the invention, the polyvalent-antigen particle further comprises a carrier portion which is coupled to an antigenic portion, optionally via a linker.

The term “linker”, as described herein, refers to any molecule(s), peptides or structures which may be used to covalently or non-covalently connect two portions of the compounds of the invention with each other. In some embodiments the linker described herein is a peptide linker which may have any size and length suitable for a given application in context of the invention. Linkers may have a length or 1-100 amino acids, preferably of 2 to 50 amino acids. A linker could be a typical 4GS linker in 2, 3, 4, 5, 6 or more repeats.

In certain embodiments, the invention relates to the composition for use of the invention, wherein the cross-link in the polyvalent-antigen particle is a chemical cross-link, such as a bis-maleimide mediated cross-link, or is a protein cross-link, such as a biotin-streptavidin mediated cross-link.

In certain embodiments, the invention relates to the polyvalent particle for use of the invention or the composition for use of the invention, wherein the pathogen-associated antigen comprises at least one agent selected from the group of nucleic acid, carbohydrate and peptide.

In certain embodiments, the invention relates to the composition for use of the invention, wherein the polyvalent-antigen particle comprises a complex of the following formula A-L-A, wherein A is a target antigen comprising portion, and wherein L is the linker of the cross link, preferably wherein L is a bismaleimide, and most preferably the complex is of the following structure (I), wherein R is a target antigen comprising portion:

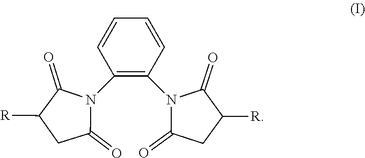

Preferably, neither the carrier portion, and optionally also not the linker, is (are) capable of eliciting an antibody-mediated immune response against the target antigen.

The carrier portion can facilitate presentation of the antigen to the immune system and improve stability of the particle.

Accordingly, the invention is at least in part based on the surprising finding that a carrier linked to the antigenic portion can improve the antigenic, pharmacologic and/or pharmacokinetic properties of the polyvalent antigen particle and therefore influence the modulation of the immune response to a target antigen as described herein.

In certain embodiments, the invention relates to the composition for use of the invention, wherein the polyvalent-antigen particle comprises a linker with a crosslink reactive group for protein conjugation.

The term “crosslink reactive group for protein conjugation”, as used herein, refers to any chemical group or structure that enables creating a link between the antigen particles described herein and a protein. Such crosslink reactive groups ant the preparation thereof a well known to the person skilled in the art (see e.g. Brinkley, M., 1992, Bioconjugate chemistry, 3 (1), 2-13; Kluger, R., & Alagic, A, 2004, Bioorganic chemistry 32.6 (2004): 451-472.; Stephanopoulos, N.; Francis, M. B., 2011, Nature Chemical Biology. 7 (12): 876-884).

The inventors found that a linker that is linked to the antigen particle described herein (e.g. the polyvalent antigen particle) and that comprises a crosslink reactive group to bind to endogenous protein in a subject can enhance the immune response (see e.g. FIGS. 8-12, Example 7, 9, 10).

In certain embodiments, the invention relates to the composition for use of the invention, wherein the polyvalent-antigen particle comprises a linker with a crosslink reactive group for stable protein conjugation.

The term “stable protein conjugation”, as used herein, refers to a covalent protein conjugation that is not an S—S binding. In some embodiments, the stable protein conjugation described herein is hydrolytically stable. In some embodiments, the stable protein conjugation described herein is an irreversible binding.

The inventors found that stable binding to endogenous proteins can enhance the immune reaction against the antigen particles described herein (Example 9).

In certain embodiments, the invention relates to the composition for use of the invention, wherein the crosslink reactive group couples to a protein with at least one selected from the group of lysine amino acid residue, cysteine residue, tyrosine residues, tryptophan residues, N-terminus and C-terminus.

In certain embodiments, the invention relates to the composition for use of the invention, wherein the crosslink reactive group is a group selected from carboxyl-to-amine reactive groups, amine-reactive groups, sulfhydryl-reactive groups, aldehyde-reactive groups and photoreactive groups.

In certain embodiments, the invention relates to the composition for use of the invention, wherein the crosslink reactive group is a group selected from carbodiimide, NHS ester, imidoester, pentafluorophenyl ester, hydroxymethyl phosphine, maleimide, haloacetyl, hydrazide, alkoxyamine, diazirine and aryl azide.

Accordingly, the invention is at least in part based on the enhancement of the immune response by binding to endogenous proteins.

In certain embodiments, the invention relates to the composition for use of the invention, wherein the polyvalent antigen particle comprises the at least two copies of the antigenic structure in spatial proximity to each other, preferably within a range of 3 nm to 20 nm.

A polyvalent-antigen particle of the invention preferably comprises the at least two copies of the antigenic structure in spatial proximity to each other, preferably within a nanometer range selected from the ranges about 1 nm to about 1000 nm, about 1 nm to about 500 nm, about 1 nm to about 100 nm, about 1 nm to about 50 nm, about 1 nm to about 20 nm or about 3 nm to about 20 nm.

Methods for measurement of spatial proximity are known to the person skilled in the art (see e.g. F. Schueder et al., 2021, Angew. Chem. Int. Ed. 2021, 60, 716; Erickson, D. et al., 2008, Microfluidics and nanofluidics, 4 (1-2), 33-52; Turkowyd, B., et al., 2016, Anal Bioanal Chem 408, 6885-6911).

The inventors found that the polyvalent particles in a certain size range are particularly effective in eliciting certain immune responses.

Accordingly, the invention is at least in part based on the surprising finding that the size of the antigen particle and/or the spatial proximity can influence the modulation of the immune response to a target antigen as described herein.

In certain embodiments, the invention relates to the composition for use of the invention, wherein the pathogen-associated antigen comprises at least one agent selected from the group of nucleic acid, carbohydrate and peptide.

Nucleic acids, carbohydrates and/or peptides are useful structures to copy or mimic antigen patterns of pathogens. Furthermore, they can be designed to elicit a specific immune response without substantial side effects.

Accordingly, the invention is at least in part based on the surprising finding that certain antigen types can influence the modulation of the immune response to a target antigen as described herein.

In certain embodiments, the invention relates to the composition for use of the invention, wherein the polyvalent antigen particle is linked to an adjuvant, preferably wherein the polyvalent particle is covalently linked to an adjuvant.

The term “adjuvant”, as used herein, refers to an agent that does not comprise the target antigen and can enhance the immune response to the antigen particles described herein. In some embodiments, the adjuvant described herein comprises at least one adjuvant selected from the group of oils (e.g., paraffin oil, peanut oil), bacterial products, saponins, cytokines (e.g., IL-1, IL-2, IL-12), squalene and IgG, preferably wherein the adjuvant comprises a free SH-group.

The inventors found that linking the antigen particles described herein to adjuvants can enhance the immune response, in particular the immune response induce by the polyvalent antibody (FIGS. 9D and E, FIG. 11, 12). This linking to adjuvants reduces the necessity of formulating the antigen particles described herein with substantially larger amounts of non-linked adjuvants. Furthermore, the adjuvants can increase the stability of the antigen particles described herein.

Accordingly, the invention is at least in part based on the finding that linking of the antigen particles described herein to adjuvants can enhance the elicited immune response.

In certain embodiments, the invention relates to the composition for use of the invention, wherein the adjuvant is IgG.

The term “IgG”, as used herein, refers to a molecule that consists of or comprises an polypeptide of the immunoglobulin G class.

Conventional adjuvants are associated with side effects (see e.g. Petrovsky, Nikolai. Drug safety 38.11 (2015): 1059-1074). The inventors found, that linking of the antigen particles described herein with IgG is useful to enhance the immune response and subsequently reducing the need necessity of formulating the antigen particles described herein with conventional adjuvants (FIGS. 9D and E, FIG. 11, 12).

Accordingly, the invention is at least in part based on the finding that linking of the antigen particles described herein to IgG can enhance the elicited immune response.

In certain embodiments, the invention relates to the composition for use of the invention, wherein treatment and/or prevention comprises at least two administration time points.

Therefore, the ingredients of the composition of the invention can be administered at different time points to achieve a certain immune modulation or can be administered repeatedly to boost achieve an enhanced effect (see FIG. 1).

Accordingly, the invention is at least in part based on the surprising finding that priming and/or boosting modulates the immune response alteration induced by the means and method of the invention.

In certain embodiments, the invention relates to the composition for use of the invention, wherein prevention comprises administering the mono/divalent antigen particle before the polyvalent antigen particle.

In certain embodiments, the invention relates to a method of prevention and/or treatment of an infection, the method comprising the steps of: 1) priming by administration of a mono/divalent antigen particle; and 2) boosting with a polyvalent antigen particle, wherein the mono/divalent antigen particle and the polyvalent antigen particle target the same antigen.

The means and methods of the various embodiments of the present invention in certain embodiments can be viewed as immunization methods for the generation of certain desired antibody responses. In this context, preferred embodiments of the inventive methods comprise a priming/boosting immunization scheme of the subject.

The term “priming” an immune response to an antigen refers to the administration to a subject with an immunogenic composition which induces a higher level of an immune response to the antigen upon subsequent administration with the same or a second composition, than the immune response obtained by administration with a single immunogenic composition.

The term “boosting” an immune response to an antigen refers to the administration to a subject with a second, boosting immunogenic composition after the administration of the priming immunogenic composition. In one embodiment, the boosting administration of the immunogenic composition is given about 2 to 27 weeks, preferably 1 to 10 weeks, more preferably 1 to 5 weeks, and most preferably about 3 weeks, after administration of the priming dose.

In some embodiments of the invention the step of priming is performed with the mono/divalent antigen particle which is composed of an antigenic portion comprising not more than one of an antigenic structure capable of inducing an antibody-mediated immune response against the target antigen, whereas the step of boosting comprises the administration of the polyvalent antigen particle which is composed of an antigenic portion comprising more than one of an antigenic structure capable of inducing an antibody-mediated immune response against the target antigen and wherein the more than one of an antigenic structures are covalently or non-covalently cross-linked. In such priming/boosting embodiment of the invention, the antigenic structure used for inducing the immune response in the priming and the boosting step is the same antigenic structure.

In some embodiments of the invention, the step of boosting may be performed with a composition of mono/divalent and polyvalent antigen particles.

Accordingly, the invention is at least in part based on the surprising finding that priming with a mono/divalent antigen particle increases the immune response to the polyvalent antigen particle.

In certain embodiments, the invention relates to the composition for use of the invention, wherein the treatment and/or prevention comprises at least two administration time points for the mono/divalent antigen particle and least two administration time points for the polyvalent antigen particle.

In certain embodiments, the invention relates to the composition for use of the invention, wherein the antibody-mediated immune response is an IgM-mediated immune response.

In certain embodiments, the invention relates to the composition for use of the invention, wherein the antibody-mediated immune response is an IgG-mediated immune response.

In certain embodiments, the invention relates to the composition for use of the invention, wherein the antibody-mediated immune response is an IgG and IgM mediated immune response.

The inventors found that the composition of the invention can selectively elicit an IgG and/or IgM-mediated immune response (FIGS. 2-4, 7).

In certain embodiments, the invention relates to the composition for use of the invention, wherein the pathogen is at least one pathogen selected from the group of parasite, bacterium and virus.

In certain embodiments, the invention relates to the composition for use of the invention, wherein the pathogen is at least one bacteria from a genus selected from the group consisting of Abiotrophia, Achromobacter, Acidaminococcus, Acidovorax, Acinetobacter, Actinobacillus, Actinobaculum, Actinomadura, Actinomyces, Aerococcus, Aeromonas, Afipia, Agrobacterium, Alcaligenes, Alloiococcus AlteromonasAmycolata, Amycolatopsis, Anaerobospirillum, Anaerorhabdus, “Anguillina”, Arachnia, Arcanobacterium, Arcobacter, Arthrobacter, Atopobium, Aureobacterium, Bacillus, Bacteroides, Balneatrix, Bartonella, Bergeyella, Bifidobacterium, Bilophila, Branhamella, Borrelia, Bordetella, Brachyspira, Brevibacillus, Brevibacterium, Brevundimonas, Brucella, Burkholderia, Buttiauxella, Butyrivibrio, Calymmatobacterium, Campylobacter, Capnocytophaga, Cardiobacterium, Catonella, Cedecea, Cellulomonas, Centipeda, Chlamydia, Chlamydophila, Chromobacterium, Chyseobacterium, Chryseomonas, Citrobacter, Clostridium, Collinsella, Comamonas, Corynebacterium, Coxiella, Cryptobacterium, Delftia, Dermabacter, Dermatophilus, Desulfomonas, Desulfovibrio, Dialister, Dichelobacter, Dolosicoccus, Dolosigranulum, Edwardsiella, Eggerthella, Ehrlichia, Eikenella, Empedobacter, Enterobacter, Enterococcus, Erwinia, Erysipelothrix, Escherichia, Eubacterium, Ewingella, Exiguobacterium, Facklamia, Filifactor, Flavimonas, Flavobacterium, Flexispira, Francisella, Fusobacterium, Gardnerella, Gemella Globicatella, Gordona, Haemophilus, Hafnia, Helicobacter, Helococcus, Holdemania, Ignavigranum, Johnsonella, Kingella, Klebsiella, Kocuria, Koserella, Kurthia, Kytococcus, Lactobacillus, Lactococcus, Lautropia, Leclercia, Legionella, Leminorella, Leptospira, Leptotrichia, Leuconostoc, Listeria, Listonella, Megasphaera, Methylobacterium, Microbacterium, Micrococcus, Mitsuokella, Mobiluncus, Moellerella, Moraxella, Morganella, Mycobacterium, Mycoplasma, Myroides, Neisseria, Nocardia, Nocardiopsis, Ochrobactrum, OeskoviaOligella, Orientia, Paenibacillus, Pantoea, Parachlamydia, Pasteurella, Pediococcus, Peptococcus, Peptostreptococcus, Photobacterium, Photorhabdus, Plesiomonas Porphyrimonas, Prevotella, Propionibacterium, Proteus, Providencia, Pseudomonas, Pseudonocardia, Pseudoramibacter, Psychrobacter, Rahnella, Ralstonia, Rhodococcus, Rickettsia, Rochalimaea, Roseomonas, Rothia, Ruminococcus, Salmonella, Selenomonas, Serpulina, Serratia, Shewenella, Shigella, Simkania, Slackia, Sphingobacterium, Sphingomonas, Spirillum, Staphylococcus, Stenotrophomonas, Stomatococcus, Streptobacillus, Streptococcus, Streptomyces, Succinivibrio, Sutterella, Suttonella, Tatumella, Tissierella, Trabulsiella, Treponema, Tropheryma, Tsakamurella, Turicella, Ureaplasma, Vagococcus, Veillonella, Vibrio, Weeksella, Wolinella, Xanthomonas, Xenorhabdus, Yersinia and Yokenella.

In certain embodiments, the invention relates to the composition for use of the invention, wherein the pathogen is at least one bacteria from the group consisting of Bacteria Actimomyces europeus, Actimomyces georgiae, Actimomyces gerencseriae, Actimomyces graevenitzii, Actimomyces israelii, Actimomyces meyeri, Actimomyces naeslundii, Actimomyces neuii neuii, Actimomyces neuii anitratus, Actimomyces odontolyticus, Actimomyces radingae, Actimomyces turicensis, Actimomyces viscosus, Arthrobacter creatinolyticus, Arthrobacter cumminsii, Arthrobacter woluwensis, Bacillus anthracis, Bacillus cereus, Bacillus circulans, Bacillus coagulans, Bacillus licheniformis, Bacillus megaterium, Bacillus myroides, Bacillus pumilus, Bacillus sphaericus, Bacillus subtilis, Bacillus thuringiensis, Borrelia afzelii, Borrelia andersonii, Borrelia bissettii, Borrelia burgdorferi, Borrelia garinii, Borrelia japonica, Borrelia lusitaniae, Borrelia tanukii, Borrelia turdi, Borrelia valaisiana Borrelia caucasica, Borrelia crocidurae, Borrelia recurrentis, Borrelia duttoni, Borrelia graingeri, Borrelia hermsii, Borrelia hispanica, Borrelia latyschewii, Borrelia mazzottii, Borrelia parkeri, Borrelia persica, Borrelia recurrentis, Borrelia turicatae, Borrelia venezuelensi, Bordetella bronchiseptica, Bordetella hinzii, Bordetella holmseii, Bordetella parapertussis, Bordetella pertussis, Bordetella trematum, Clostridium absonum, Clostridium argentinense, Clostridium baratii, Clostridium bifermentans, Clostridium beijerinckii, Clostridium butyricum, Clostridium cadaveris, Clostridium carnis, Clostridium celatum, Clostridium clostridioforme, Clostridium cochlearium, Clostridium cocleatum, Clostridium fallax, Clostridium ghonii, Clostridium glycolicum, Clostridium haemolyticum, Clostridium hastiforme, Clostridium histolyticum, Clostridium indolis, Clostridium innocuum, Clostridium irregulare, Clostridium leptum, Clostridium limosum, Clostridium malenominatum, Clostridium novyi, Clostridium oroticum, Clostridium paraputrificum, Clostridium piliforme, Clostridium putrefasciens, Clostridium ramosum, Clostridium septicum, Clostridium sordelii, Clostridium sphenoides, Clostridium sporogenes, Clostridium subterminale, Clostridium symbiosum, Clostridium tertium, Clostridium tetani, Escherichia coli, Escherichia fergusonii, Escherichia hermanii, Escherichia vulneris, Enterococcus avium, Enterococcus casseliflavus, Enterococcus cecorum, Enterococcus dispar, Enterococcus durans, Enterococcus faecalis, Enterococcus faecium, Enterococcus flavescens, Enterococcus gallinarum, Enterococcus hirae, Enterococcus malodoratus, Enterococcus mundtii, Enterococcus pseudoavium, Enterococcus raffinosus, Enterococcus solitarius, Haemophilus aegyptius, Haemophilus aphrophilus, Haemophilus paraphrophilus, Haemophilus parainfluenzae, Haemophilus segnis, Haemophilus ducreyi, Haemophilus influenzae, Klebsiella ornitholytica, Klebsiella oxytoca, Klebsiella planticola, Klebsiella pneumoniae, Klebsiella ozaenae, Klebsiella terrigena, Lysteria ivanovii, Lysteria monocytogenes, Mycobacterium abscessus, Mycobacterium africanum, Mycobacterium alvei, Mycobacterium asiaticum, Mycobacterium aurum, Mycobacterium avium, Mycobacterium bohemicum, Mycobacterium bovis, Mycobacterium branderi, Mycobacterium brumae, Mycobacterium celatum, Mycobacterium chelonae, Mycobacterium chubense, Mycobacterium confluentis, Mycobacterium conspicuum, Mycobacterium cookii, Mycobacterium flavescens, Mycobacterium fortuitum, Mycobacterium gadium, Mycobacterium gastri, Mycobacterium genavense, Mycobacterium gordonae, Mycobacterium goodii, Mycobacterium haemophilusobacterium hassicum, Mycobacterium intracellulare, Mycobacterium interjectum, Mycobacterium heidelberense, Mycobacterium kansasii, Mycobacterium lentiflavum, Mycobacterium leprae, Mycobacterium malmoense, Mycobacterium marinum, Mycobacterium microgenicum, Mycobacterium microti, Mycobacterium mucogenicum, Mycobacterium neoaurum, Mycobacterium nonchromogenicum, Mycobacterium peregrinum, Mycobacterium phlei, Mycobacterium scrofulaceum, Mycobacterium shimoidei, Mycobacterium simiae, Mycobacterium smegmatis, Mycobacterium szulgai, Mycobacterium terrae, Mycobacterium thermoresistabile, Mycobacterium triplex, Mycobacterium triviale, Mycobacterium tuberculosis, Mycobacterium tusciae, Mycobacterium ulcerans, Mycobacterium vaccae, Mycobacterium wolinskyi, Mycobacterium xenopi, Mycoplasma buccale, Mycoplasma faucium, Mycoplasma fermentans, Mycoplasma genitalium, Mycoplasma hominis, Mycoplasma lipophilum, Mycoplasma orale, Mycoplasma penetrans, Mycoplasma pirum, Mycoplasma pneumoniae, Mycoplasma primatum, Mycoplasma salivarium, Mycoplasma spermatophilum, Pseudomonas aeruginosa, Pseudomonas alcaligenes, Pseudomonas chlororaphis, Pseudomonas fluorescens, Pseudomonas luteola. Pseudomonas mendocina, Pseudomonas monteilii, Pseudomonas oryzihabitans, Pseudomonas pertocinogena, Pseudomonas pseudalcaligenes, Pseudomonas putida, Pseudomonas stutzeri, Rickettsia africae, Rickettsia akari, Rickettsia australis, Rickettsia conorii, Rickettsia felis, Rickettsia honei, Rickettsia japonica, Rickettsia mongolotimonae, Rickettsia prowazekii, Rickettsia rickettsiae, Rickettsia sibirica, Rickettsia slovaca, Rickettsia typhi, Salmonella choleraesuis choleraesuis, Salmonella choleraesuis arizonae, Salmonella choleraesuis bongori, Salmonella choleraesuis diarizonae, Salmonella choleraesuis houtenae, Salmonella choleraesuis indica, Salmonella salamae, Salmonella enteritidis, Salmonella typhi, Salmonella typhimurium, Shigella boydii, Shigella dysentaeriae, Shigella flexneri, Shigella sonnei, Staphylococcus aureus, Staphylococcus auricularis, Staphylococcus capitis capitis, Staphylococcus c. ureolyticus, Staphylococcus caprae, Staphylococcus aureus, Staphylococcus cohnii cohnii, Staphylococcus c. urealyticus, Staphylococcus epidermidis, Staphylococcus equorum, Staphylococcus gallinarum, Staphylococcus haemolyticus, Staphylococcus hominis hominis, Staphylococcus h. novobiosepticius, Staphylococcus hyicus, Staphylococcus intermedius, Staphylococcus lugdunensis, Staphylococcus pasteuri, Staphylococcus saccharolyticus, Staphylococcus saprophyticus, Staphylococcus schleiferi schleiferi, Staphylococcus s. coagulans, Staphylococcus sciuri, Staphylococcus simulans, Staphylococcus warneri, Staphylococcus xylosus, Streptococcus agalactiae, Streptococcus canis, Streptococcus dysgalactiae dysgalactiae, Streptococcus dysgalactiae equisimilis, Streptococcus equi equi, Streptococcus equi zooepidemicus, Streptococcus iniae, Streptococcus porcinus, Streptococcus pyogenes, Streptococcus anginosus, Streptococcus constellatus constellatus, Streptococcus constellatus pharyngidis, Streptococcus intermedius, Streptococcus mitis, Streptococcus oralis, Streptococcus sanguinis, Streptococcus cristatus, Streptococcus gordonii, Streptococcus parasanguinis, Streptococcus salivarius, Streptococcus vestibularis, Streptococcus criceti, Streptococcus mutans, Streptococcus ratti, Streptococcus sobrinus, Streptococcus acidominimus, Streptococcus bovis, Streptococcus equinus, Streptococcus pneumoniae, Streptococcus suis, Vibrio alginolyticus, V, carchariae, Vibrio cholerae, C. cincinnatiensis, Vibrio damsela, Vibrio fluvialis, Vibrio furnissii, Vibrio hollisae, Vibrio metschnikovii, Vibrio mimicus, Vibrio parahaemolyticus, Vibrio vulnificus, Yersinia pestis, Yersinia aldovae, Yersinia bercovieri, Yersinia enterocolitica, Yersinia frederiksenii, Yersinia intermedia, Yersinia kristensenii, Yersinia mollaretii, Yersinia pseudotuberculosis and/or Yersinia rohdei.

In certain embodiments, the invention relates to the composition for use of the invention, wherein the pathogen is Malaria (p. falciparum).

In certain embodiments, the invention relates to the composition for use of the invention, wherein the pathogen is M. tuberculosis.

In certain embodiments, the invention relates to the composition for use of the invention, wherein the pathogen is selected from the group of multiresistant bacteria (e.g. S. aureus).

In certain embodiments, the invention relates to the polyvalent particle for use of the invention or the composition for use of the invention, wherein the infection is a viral infection. Preferably, in this embodiment, the pathogen is a virus.

In some embodiments, the viral infection described herein is an infection of a virus selected from the group of adenoviridae, anelloviridae, arenaviridae, astroviridae, bunyaviridae, bunyavirus, caliciviridae, coronaviridae, filoviridae, flaviviridae, hepadnaviridae, herpesviridae, orthomyxoviridae, papillomaviridae, paramyxoviridae, parvoviridae, picornaviridae, pneumoviridae, polyomaviridae, poxviridae, reoviridae, retroviridae, rhabdoviridae, rhabdovirus, and togaviridae. In some embodiments, the viral infection described herein is an infection of an RNA virus. In some embodiments, the viral infection described herein is an infection of an RNA virus selected from the group Amalgaviridae, Birnaviridae, Chrysoviridae, Cystoviridae, Endornaviridae, Hypoviridae, Megabirnaviridae, Partitiviridae, Picobirnaviridae, Reoviridae, Totiviridae, Quadriviridae, Botybirnavirus, Unassigned dsRNA viruses, Arteriviridae, Coronaviridae (includes inter alia Coronavirus, SARS-COV), Mesoniviridae, Roniviridae, Dicistroviridae, Iflaviridae, Marnaviridae, Picornaviridae, Secoviridae, Alphaflexiviridae, Betaflexiviridae, Gammaflexiviridae, Tymoviridae, Alphatetraviridae, Alvernaviridae, Astroviridae, Barnaviridae, Benyviridae, Botourmiaviridae, Bromoviridae, Caliciviridae, Carmotetraviridae, Closteroviridae, Flaviviridae, Fusariviridae, Hepeviridae, Hypoviridae, Leviviridae, Luteoviridae, Polycipiviridae, Narnaviridae, Nodaviridae, Permutotetraviridae, Potyviridae, Sarthroviridae, Statovirus, Togaviridae, Tombusviridae, Virgaviridae, Unassigned genera positive-sense ssRNA viruses, Qinviridae, Aspiviridae, Chuviridae, Bornaviridae, Filoviridae, Mymonaviridae, Nyamiviridae, Paramyxoviridae, Pneumoviridae, Rhabdoviridae, Sunviridae, Anphevirus, Arlivirus, Chengtivirus, Crustavirus, Wastrivirus, Yueviridae, Arenaviridae, Cruliviridae, Feraviridae, Fimoviridae, Hantaviridae, Jonviridae, Nairoviridae, Peribunyaviridae, Phasmaviridae, Phenuiviridae, Tospoviridae, Tilapineviridae, Amnoonviridae, Orthomyxoviridae, Satellite viruses (including inter alia, Sarthroviridae, Albetovirus, Aumaivirus, Papanivirus, Virtovirus, Chronic bee paralysis virus), Retroviridae, Metaviridae, and Pseudoviridae.

In some embodiments, described herein is a virus selected from the group of Adeno-associated virus, Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Cosavirus A, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, Epstein-Barr virus, European bat lyssavirus, GB virus C/Hepatitis G virus, Hantaan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus 68, Human enterovirus 70, Human herpesvirus 1, Human herpesvirus 2, Human herpesvirus 6, Human herpesvirus 7, Human herpesvirus 8, Human immunodeficiency virus, Human papillomavirus 1, Human papillomavirus 2, Human papillomavirus 16, Human papillomavirus 18,Human parainfluenza, Human parvovirus B19, Human respiratory syncytial virus, Human rhinovirus, Human SARS coronavirus, Human spumaretrovirus, Human T-lymphotropic virus, Human torovirus, Influenza A virus, Influenza B virus, Influenza C virus, Isfahan virus, JC polyomavirus, Japanese encephalitis virus, Junin arenavirus, KI Polyomavirus, Kunjin virus, Lagos bat virus, Lake Victoria marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norwalk virus, O′nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever sicilian virus, Sapporo virus, SARS coronavirus 2, Semliki forest virus, Seoul virus, Simian foamy virus, Simian virus 5, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus and/or Zika virus.

In certain embodiments, the invention relates to the composition for use of the invention, wherein the pathogen is HHV-3.

In certain embodiments, the invention relates to the composition for use of the invention, wherein the pathogen is HIV-1.

In some embodiments, the virus described herein is a variant having an at least 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% sequence identity to the viral genome sequence of at last one virus described herein.

In certain embodiments, the invention relates to the polyvalent particle for use of the invention or the composition for use of the invention, wherein the viral infection is a coronavirus infection. Preferably, in this embodiment, the pathogen is a corona virus.

Within the present invention, the Coronavirus may in particular be of the genus α-CoV, β-CoV, γ-CoV or δ-CoV. More particularly, the Coronavirus may be selected from the group consisting of Human coronavirus OC43 (HCoV-OC43), Human coronavirus HKU1 (HCoV-HKU1), Human coronavirus 229E (HCoV-229E), Human coronavirus NL63 (HCoV-NL63, New Haven coronavirus), Middle East respiratory syndrome-related coronavirus (MERS-COV or “novel coronavirus 2012”), Severe acute respiratory syndrome coronavirus (SARS-COV or “SARS-classic”), and Severe acute respiratory syndrome coronavirus 2 (SARS-COV-2 or “novel coronavirus 2019”).

In certain embodiments, the invention relates to the polyvalent particle for use of the invention or the composition for use of the invention, wherein the coronavirus infection is a SARS-COV-2 infection. Preferably, in this embodiment, the pathogen is SARS-CoV-2.

In some embodiments, the SARS-COV-2 described herein is a SARS-COV-2 variant. In some embodiments, the SARS-COV-2 variant described herein is a SARS-COV-2 variant selected from the group of Lineage B.1.1.207, Lineage B.1.1.7, Cluster 5, 501.V2 variant, Lineage P.1, Lineage B.1.429/CAL.20C, Lineage B.1.427, Lineage B.1.526, Lineage B.1.525, Lineage B.1.1.317, Lineage B.1.1.318, Lineage B.1.351, Lineage B.1.617, Lineage B.1.617.2 and Lineage P.3. In some embodiments, the SARS-COV-2 variant described herein is a SARS-COV-2 variant described by a Nextstrain clade selected from the group 19A, 20A, 20C, 20G, 20H, 20B, 20D, 20F, 20I, and 20E.

In some embodiments, the SARS-COV-2 variant described herein is a SARS-COV-2 variant selected from the group of Alpha, Delta, Beta, Gamma, Eta, lota, Kappa, Lambda. In some embodiments, the SARS-COV-2 virus described herein is a SARS-CoV-2 variant comprising at least one mutation selected from the group of N440K, L452R, S477G/N, E484Q, E484K, N501Y, D614G, P681H, P681R and A701V. In some embodiments, the SARS-COV-2 variant described herein is a SARS-COV-2 variant or a hybrid derived from the variants described herein.

The invention provides the means and methods to induce an immune response against SARS-COV-2, e.g., prior to exposure (FIG. 1). Therefore, the invention provides a novel way to produce vaccines against SARS-COV-2. By replacing the presented antigen (i.e. RBD in the example) with an antigen of another pathogen/virus/variant, the means and methods provided herein can be used for the prevention of any other pathogen. The invention may also be used to boost an insufficient immune response during an infection and may therefore also be used for treatment rather than prevention.

In certain embodiments, the invention relates to the composition for use of the invention, wherein the pathogen-associated antigen comprises an amino acid sequence derived from the corona virus spike protein, such as a receptor binding domain (RBD) sequence, preferably the complete RBD sequence, a fragment thereof, or a sequence a sequence comprising at least 80%, at least 85%, at least 90% or at least 95% sequence identity to the amino acid sequence of the SARS-COV-2 RBD amino acid sequence (SEQ ID NO: 1).

In certain embodiments, the invention relates to a method of treatment, the method comprising the steps of (i) administering to a subject: a mono/divalent antigen particle, comprising an antigenic portion comprising one or two antigenic structures capable of inducing an antibody-mediated immune response against a target antigen, wherein the target antigen is a pathogen-associated antigen, and (ii) administering to a subject: a polyvalent antigen particle comprising an antigenic portion comprising more than two antigenic structures capable of inducing an antibody-mediated immune response against the target antigen and wherein the more than two antigenic structures are cross-linked, wherein the target antigen is a pathogen associated.

In certain embodiments, the invention relates to a method of treatment, the method comprising the steps of (i) administering to a subject: a polyvalent antigen particle comprising an antigenic portion comprising more than two antigenic structures capable of inducing an antibody-mediated immune response against the target antigen and wherein the more than two antigenic structures are cross-linked, wherein the target antigen is a pathogen-associated antigen and (ii) administering to a subject: a mono/divalent antigen particle, comprising an antigenic portion comprising one or two antigenic structures capable of inducing an antibody-mediated immune response against a target antigen, wherein the target antigen is a pathogen-associated antigen.

In certain embodiments, the invention relates to a method of eliciting and/or modulating a humoral and/or B-cell-mediated immune response against a pathogen-associated target antigen, the method comprising contacting one or more B-cells with a combination comprising: (i) a mono/divalent antigen particle, comprising an antigenic portion comprising one or two antigenic structures capable of inducing an antibody-mediated immune response against a target antigen, wherein the target antigen is a pathogen-associated antigen, and (ii) a polyvalent antigen particle comprising an antigenic portion comprising more than two antigenic structures capable of inducing an antibody-mediated immune response against the target antigen and wherein the more than two antigenic structures are cross-linked, wherein the target antigen is a pathogen-associated antigen.

In certain embodiments, the invention relates to a method for producing an antibody that binds to a pathogen-associated antigen comprising the steps of: (1) administration of: (i) a mono/divalent and/or divalent antigen particle, comprising an antigenic portion comprising one or two antigenic structures capable of inducing an antibody-mediated immune response against a target antigen, wherein the target antigen is a pathogen-associated antigen, and (ii) a polyvalent antigen particle and/or a precursor thereof, wherein the polyvalent antigen particle comprises an antigenic portion comprising more than two antigenic structures capable of inducing an antibody-mediated immune response against the target antigen and wherein the more than two antigenic structures are cross-linked, wherein the target antigen is a pathogen-associated antigen, to a subject and/or a cell capable of producing antibodies; and (2) isolating an antibody from the subject and/or cell capable of producing antibodies, wherein the antibody binds to the target antigen.

The term “subject”, as used herein, refers to an animal, such as a mammal, including a primate (such as a human a non-human primate, e.g. a monkey, and a chimpanzee), a non-primate (such as a cow a pig, a camel, a llama, a horse, a goat, a rabbit, a sheep, a hamster, a guinea pig, a cat, a dog, a rat, a mouse, a horse and a whale), or a bird (e.g. a duck or a goose). In some embodiments, the subject described herein is a non-human animal.

The “cell capable of producing antibodies” is preferably a b-cell, a hybridoma cell, a myeloma cell and/or a cell genetically modified to produce antibodies. In some embodiments, the cell capable of producing antibodies described herein is a cell of a cell line.

Methods for the isolation of antibodies are known to the person skilled in the art (see e.g. Huang J, Doria-Rose N A, et al., 2013, Nat Protoc. Oct; 8 (10): 1907-15). Any method known to the person skilled in the art can be used to isolate the antibody from the subject and/or cell. In some embodiments, isolating an antibody as described herein comprises at least one method selected from the group of physicochemical fractionation, class-specific affinity and antigen-specific affinity.

In certain embodiments, the invention relates to a polynucleotide encoding: (i) a mono/divalent and/or divalent antigen particle, comprising an antigenic portion comprising one or two antigenic structures capable of inducing an antibody-mediated immune response against a target antigen, wherein the target antigen is a pathogen-associated antigen, and (ii) a polyvalent antigen particle and/or a precursor thereof, wherein the polyvalent antigen particle comprises an antigenic portion comprising more than two antigenic structures capable of inducing an antibody-mediated immune response against the target antigen and wherein the more than two antigenic structures are cross-linked, wherein the target antigen is a pathogen-associated antigen; for use in the treatment and/or prevention of an infection.

The term “polynucleotide”, as used herein, refers to a nucleic acid sequence. The nucleic acid sequence may be a DNA or a RNA sequence, preferably the nucleic acid sequence is a DNA sequence. The polynucleotides of the present invention shall be provided, preferably, either as an isolated polynucleotide (i.e. isolated from its natural context) or in genetically modified form. An isolated polynucleotide as referred to herein also encompasses polynucleotides which are present in cellular context other than their natural cellular context, i.e. heterologous polynucleotides. The term polynucleotide encompasses single as well as double stranded polynucleotides. Moreover, comprised are also chemically modified polynucleotides including naturally occurring modified polynucleotides such as glycosylated or methylated polynucleotides or artificial modified one such as biotinylated polynucleotides.

In certain embodiments, the invention relates to a vector comprising the polynucleotide for use of the invention.

The term “vector”, as used herein, refers to a nucleic acid molecule, capable transferring or transporting another nucleic acid molecule. The transferred nucleic acid is generally linked to, i.e., inserted into, the vector nucleic acid molecule. A vector may include sequences that direct autonomous replication in a cell or may include sequences sufficient to allow integration into host cell DNA. Useful vectors include, for example, plasmids (e.g., DNA plasmids or RNA plasmids), transposons, cosmids, bacterial artificial chromosomes, and viral vectors.

The polynucleotide and/or the vector described herein may be used for producing the antigen particles described herein and/or parts thereof.

“a,” “an,” and “the” are used herein to refer to one or to more than one (i.e., to at least one, or to one or more) of the grammatical object of the article.

“or” should be understood to mean either one, both, or any combination thereof of the alternatives. “and/or” should be understood to mean either one, or both of the alternatives.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

The terms “include” and “comprise” are used synonymously. “preferably” means one option out of a series of options not excluding other options. “e.g.” means one example without restriction to the mentioned example. By “consisting of” is meant including, and limited to, whatever follows the phrase “consisting of.”

The terms “about” or “approximately”, as used herein, refer to “within 20%”, more preferably “within 10%”, and even more preferably “within 5%”, of a given value or range.

Reference throughout this specification to “one embodiment,” “an embodiment,” “a particular embodiment,” “a related embodiment,” “a certain embodiment,” “an additional embodiment,” “a specific embodiment” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments. It is also understood that the positive recitation of a feature in one embodiment, serves as a basis for excluding the feature in a particular embodiment.

Unless otherwise defined, all technical and scientific terms used herein have the same meaning as commonly understood by one of ordinary skill in the art to which this invention pertains. In case of conflict, the present specification, including definitions, will control. In addition, the materials, methods, and examples are illustrative only and not intended to be limiting.

The general methods and techniques described herein may be performed according to conventional methods well known in the art and as described in various general and more specific references that are cited and discussed throughout the present specification unless otherwise indicated. See, e.g., Sambrook et al., Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992), and Harlow and Lane Antibodies: A Laboratory Manual, Cold Spring Harbor Laboratory Press, Cold Spring Harbor, N.Y. (1990).

While aspects of the invention are illustrated and described in detail in the figures and foregoing description, such illustration and description are to be considered illustrative or exemplary and not restrictive. It will be understood that changes and modifications may be made by those of ordinary skill within the scope and spirit of the following claims. In particular, the present invention covers further embodiments with any combination of features from different embodiments described above and below.

### BRIEF DESCRIPTION OF FIGURES

FIG. 1: Antibody responses after immunization with SARS-COV-2-derived RBD. Mice were pre-treated as indicated two weeks before immunization. Subsequently, the mice were immunized at day 1 and day 21. Serum was collected at day 28 after immunization concentrations and used in ELISA to determine Ig concentration.

FIG. 2: No antibody responses after immunization with native RBD while complex RBD induces weak response.

A. Schematic illustration of SARS-COV-2 Spike protein: Receptor-binding domain (RBD) which interacts with human angiotensin converting enzyme 2 (ACE2) and thereby mediates entry of viral particles into the host cell was described as a target for neutralizing antibodies.

B. Native RBD (˜27 kDa) was produced in HEK293-6E cells, biotinylated and complexed by addition of streptavidin (SAV), samples were separated (here: under non-reducing conditions) on a 10% Coomassie gel. RBD forms self-aggregates that can be dissolved by reducing disulphide bonds with b-mercaptoethanol.

C. Schematic overview of immunization procedure: WT mice were either control immunized (CI), immunized i. p. with 50 μg of native RBD (nRBD), RBD complexed with streptavidin (cRBD) in presence of CpG-ODN #1826 or obtained repeated injections of native RBD (6 i.p. administrations of 50 μg each in absence of adjuvant, within 14 days). Immunization was boosted on day 21 in CI, nRBD- and cRBD-immunized mice with the same vaccination composition used for primary immunization.

D. Blood was taken from immunized mice (described in C) at the indicated time points and RBD-specific IgM and IgG was measured by ELISA. Immunization complexed RBD induces only a weak antibody response, detectable only after boost. Repeated exposure to native RBD also induces antibody response comparable to that induced by cRBD.

FIG. 3: Combining repeated nRBD treatment with cRBD immunization results in robust antibody responses

A. Schematic overview of immunization procedure

B. Blood was collected from immunized mice (described in A) at the indicated time points.

FIG. 4: The effect of repeated nRBD treatment may last extended time

A. Schematic overview of immunization procedure. B. Blood was collected from immunized mice (described in A) at the indicated time points. RBD-specific IgM and IgG was determined by ELISA.

Pre-treatment with native RBD primes for efficient antibody responses even if the primary immune response is delayed by 5 weeks.

FIG. 5: High antibody titer is required for virus neutralization in vitro

A. Concentration of RBD-specific IgM (left), IgG (middle) and total Ig (right) determined by ELISA in samples used for neutralization assay. Sera were collected at d28 one week after boost.

B.-C. The neutralizing potential was compared amongst sera collected after cRBD immunization in the group of PBS-(−PT) and nRBD-pretreated (+PT) mice. Neutralizing capacity correlates with concentration of total RBD-specific Ig.

FIG. 6: Strong early antibody response by IgD-deficient mice

FIG. 7: Mimicking immune complexes by random crosslinking of RBD results in robust antibody responses

A. Native RBD (˜27 kDa) was produced in HEK293-6E cells and chemically cross-linked by addition of maleimide (cRBD*MM). Samples were separated (here: under reducing conditions) on a 10% Coomassie gel.

B. Schematic overview of immunization procedure.

C. Blood was collected from immunized mice (described in B) at the indicated time points. RBD-specific IgM and IgG was measured in both groups by ELISA and compared to titers measured in CI mice.

FIG. 8: Mimicking immune complexes by chemical crosslinking of RBD results in robust antibody responses

A. Concentration of RBD-specific IgM (left), IgG (middle) and total Ig (right) determined by ELISA in samples used for neutralization assay.

B-C. The neutralizing potential measured in sera from mice immunized with CRBD*MM. Results were compared to neutralizing capacities determined in mice immunized with cRBD-SAV after RBD-pre-treatment.

IgM is not exclusively required to achieve virus neutralization→can also be achieved by samples that contain mainly IgG. Higher concentrations of RBD-specific total Ig correlates with potent neutralization capacity. cRBD MM: complexed RBD with maleimide (MM)

FIG. 9: Activated antigen forms IgG complexes that boost immune responses

A. Schematic illustration of the SARS-COV-2 spike protein with localization of the receptor binding domain (RBD).

B. Reaction scheme of chemical cross-linking. At pH 6.5-7.5 reactive groups of 1,2-phenylene-bis-maleimide

(marked in red) undergo oxidation with sulfhydryl-groups on cysteine residues of proteins to form a stable thioether linkage.

C. Coomassie staining for RBD complexed by 1,2-phenylene-bis-maleimide (bismale). RBD indicates native RBD without crosslinking.

D. & E. Immunization with RBD

FIG. 10: Activated antigen forms IgG complexes that boost immune responses

A. Schematic illustration of the SARS-COV-2 spike protein with localization of the receptor binding domain (RBD).

B. Reaction scheme of chemical cross-linking. At pH 6.5-7.5 reactive groups of 1,2-phenylene-bis-maleimide (marked in red) undergo oxidation with sulfhydryl-groups on cysteine residues of proteins to form a stable thioether linkage.

C. Analysis of 1,2-phenylene-bis-maleimide-complexed RBD under reducing conditions on a 10% SDS page by Coomassie staining. RBD indicates native RBD in absence of 1,2-phenylene-bis-maleimide. RBD* was complexed with 20 μg 1,2-phenylene-bis-maleimide per 100 μg of RBD, while RBD** indicates complexation with 100 μg 1,2-phenylene-bis-maleimide per 100 μg of RBD.

D. WT mice were immunized either with RBD* or RBD** that was generated as described using different amounts of the crosslinking agent (maleimide) in “C”.

FIG. 11: Generation of antigen (Ag) complexes by biotinylation and subsequent incubation with streptavidin (SAV).

A. The biotin-SAV complex formation require additional steps including biotinylation and SAV.

B. The biochemical activation of the antigen in the presence of IgG is simpler. MM, maleimide crosslinking.

Antigen complexes possessing a reactive maleimide group form complexes with autoantigens and this boosts the immune response.

The invention will be further described in the following examples, which do not limit the scope of the invention described in the claims.

FIG. 12: Activated antigen forms IgG complexes that boost immune responses

A. Schematic illustration of the SARS-COV-2 spike protein with localization of the receptor binding domain (RBD).

B. Reaction scheme of chemical cross-linking. At pH 6.5-7.5 reactive groups of 1,2-phenylene-bis-maleimide undergo oxidation with sulfhydryl-groups on cysteine residues of proteins to form a stable thioether linkage.

C. Coomassie staining for RBD complexed by 1,2-phenylene-bis-maleimide (bismale). RBD indicates native RBD without crosslinking.

D. & E. Immunization with RBD

### EXAMPLES

### Example 1: Immunization Scheme

Virus-derived peptides (Peptides&Elephants, Berlin) (SEQ ID NO: 2, SEQ ID NO: 3) were dissolved according to their water solubility in pure water or 1% dimethylsulfoxide (DMSO). The virus-derived peptides (SEQ ID NO: 2, SEQ ID NO: 3) were coupled to Biotin or KLH, respectively. An amount of 1 mg was purchased and dissolved in a volume of 1 ml. 10 to 50 μg of KLH-coupled peptide were used for immunization of mice via intraperitoneal injection.

The impact of the immunization concept of the invention with regard to vaccine design was tested using pathogen-specific antigens derived from SARS-COV-2 coronavirus causing COVID-19. During infection, SARS-COV-2 coronavirus binds via the receptor-binding domain (RBD) to angiotensin-converting enzyme 2 (ACE2) on the host cell surface. Thus, triggering antibody responses blocking the RBD/ACE2 interaction is considered to be key for preventing coronavirus infection. Thus, the inventors used RBD from SARS-COV-2 to the role of antigen form in immune responses during immunization.

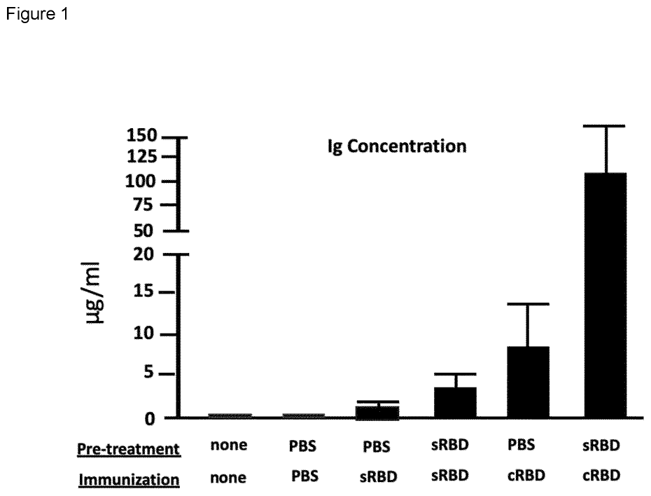

It was found that immunization with complex RBD (cRBD) (For complexation with streptavidin and biotinylated RBD were used at a ratio of 4:1. For complexation with 1,2-phenylen-bis-maleimide with a minimum of 20 μg 1,2-PBM per 100 μg RBD) induces a stronger IgG immune response as compared with soluble RBD (sRBD). For production of RBD, an expression vector encoding hexahistidine-tagged version of RBD was transiently transfected into HEK293-6E cells (Amanat, F., et al., 2020, Nature medicine, 26 (7), 1033-1036). Soluble RBD was purified from the supernatant 5 days after transfection by nickel-based immobilized metal affinity chromatography (TaKaRa)). However, the antibody concentration was not sufficient to allow virus neutralization using in-vitro infection experiments (see e.g. FIGS. 1-8). Hence, it was tested whether pretreating the mice with sRBD prior to immunization boosts immune responses. In fact, pre-treatment of the mice with soluble RBD two weeks prior to immunizations resulted in greatly augmented immune response (FIG. 1). Importantly, the serum of the pretreated mice showed an enormously high capacity to prevent SARS-COV-2 infection in vitro.

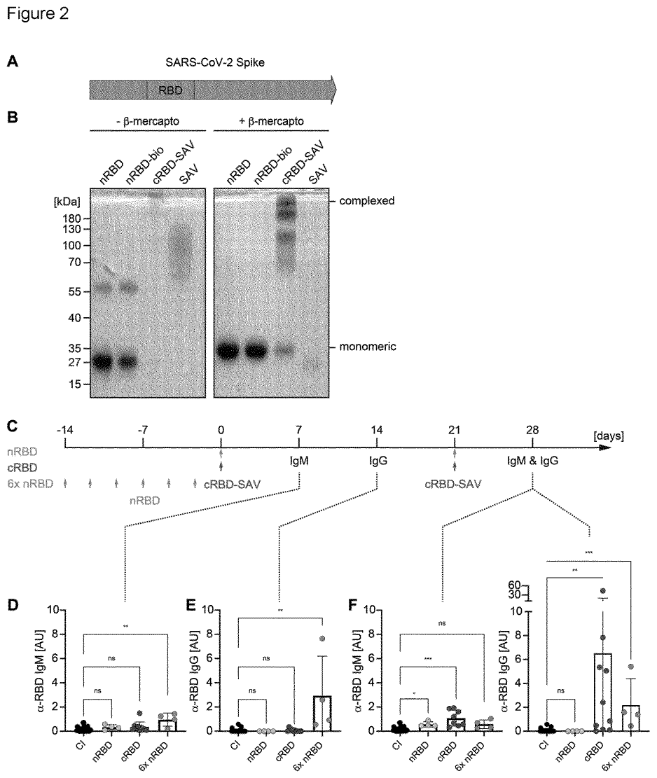

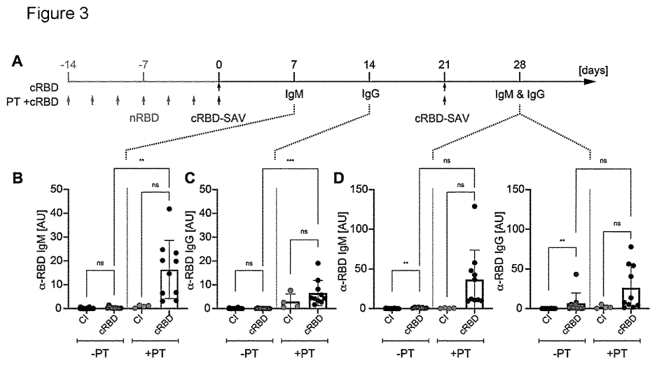

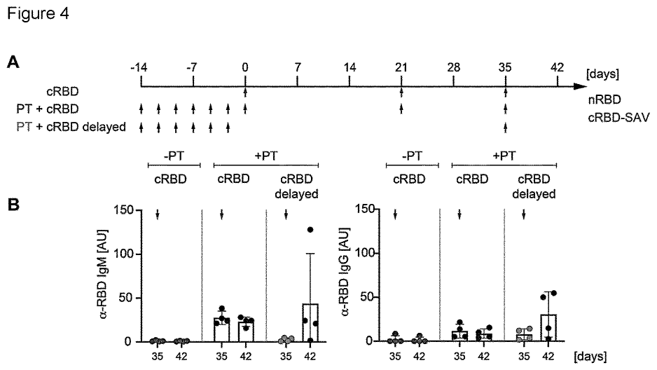

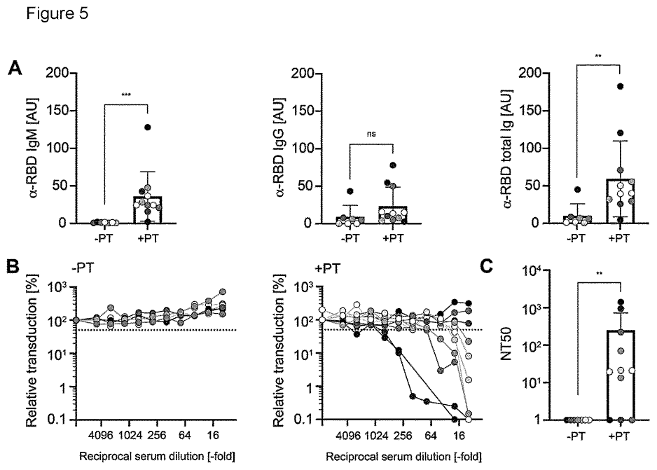

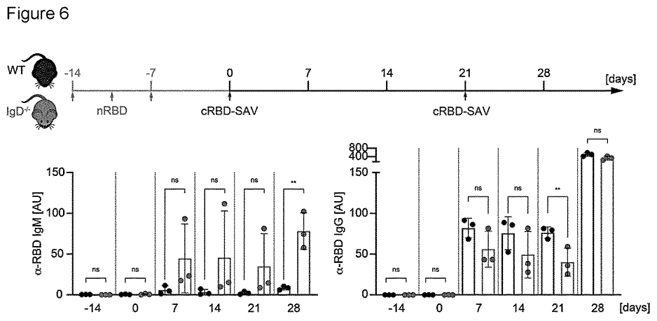

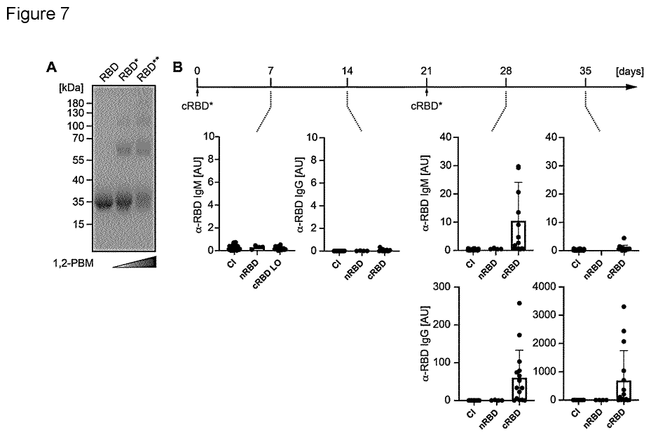

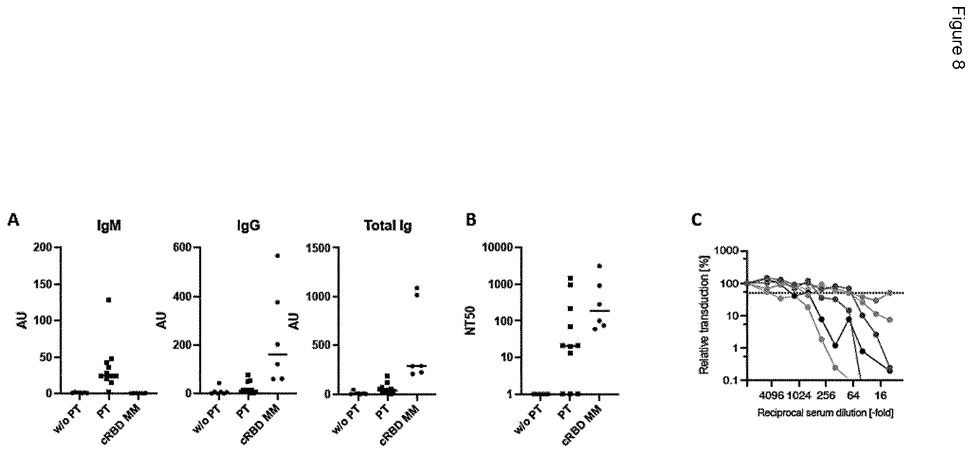

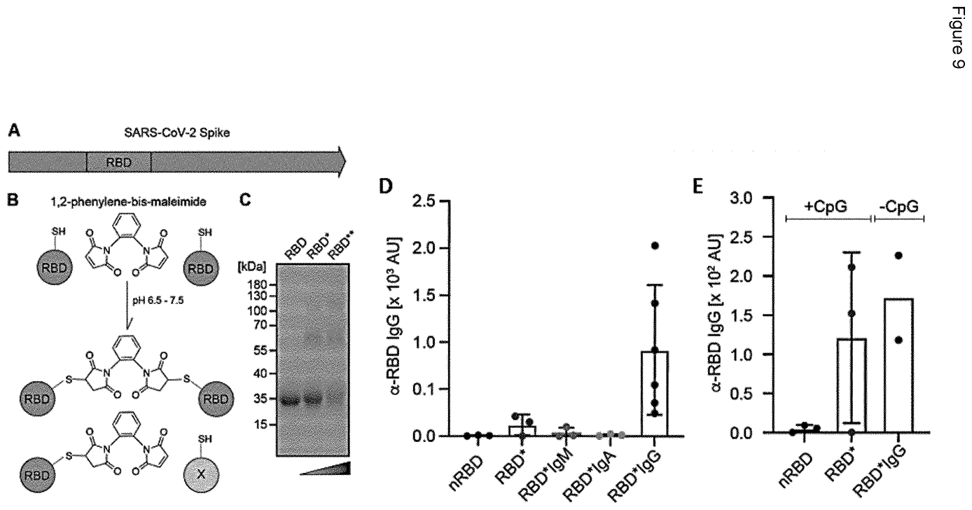

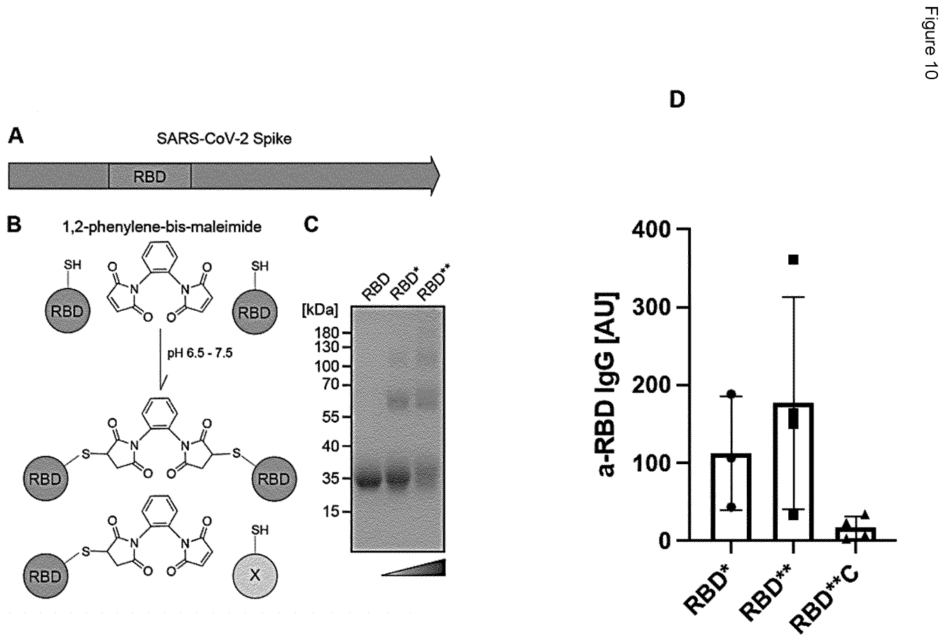

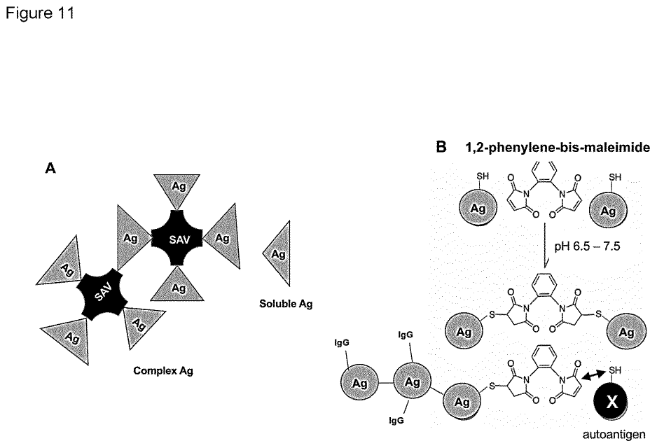

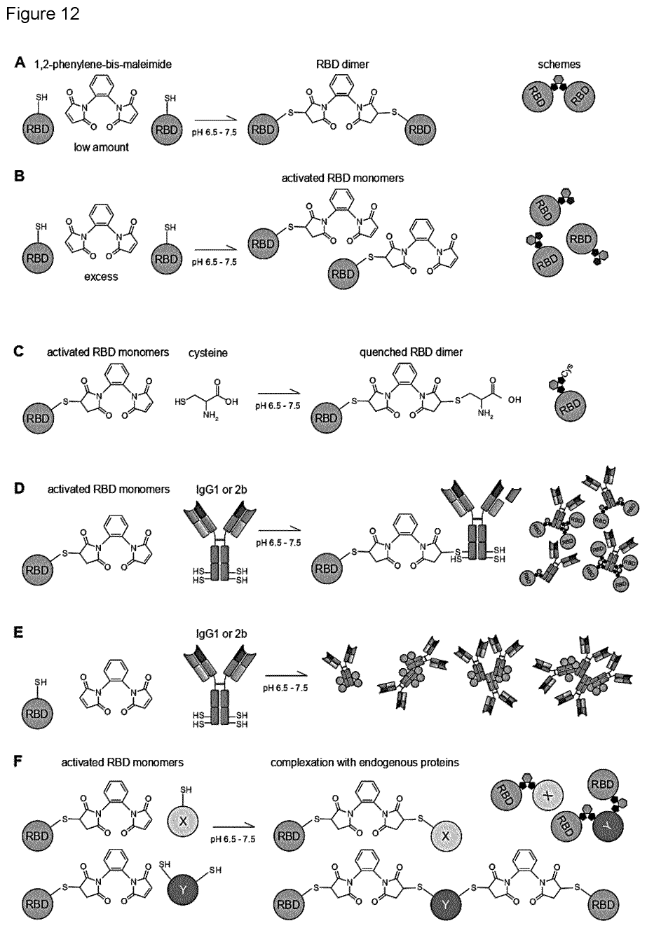

Moreover, it was found that different ratios of sRBD to cRBD in the composition of the immunization cocktail result in different ratios of immunoglobulin isotypes (i.e. IgG to IgM) which allow refined control of immune responses after immunization (see e.g. FIG. 2-8).

### Example 2 Antibody Responses after Multiple Injections of Native RBD or Complex RBD

During infection, SARS-COV-2 coronavirus binds via the receptor-binding domain (RBD) to angiotensin-converting enzyme 2 (ACE2) on the host cell surface and this binding seems to be a critical step for virus infection. Consequently, triggering antibody responses blocking the RBD/ACE2 interaction is considered to be key for preventing coronavirus infection. Therefore, we generated recombinant RBD from SARS-COV-2 and assessed the role of antigen forms in immune responses during immunization. We found that native RBD (nRBD) forms dimers under non-reducing conditions and that after biotinylating higher molecular complexes of RBD (cRBD) can be formed (FIG. 2A). Typical immunization by injecting nRBD at d0 (primary immunization) and d21 (secondary immunization or boost) failed to induce reliable antibody response while cRBD was able to induce detectable antibody responses at d28, one week after secondary immunization (FIG. 2B&C). Interestingly, 6 times repeated injection of nRBD over two weeks was also able to induce a detectable immune response (FIG. 2D). In summary, these data suggest that immunization with multivalent complex RBD or multiple injections of nRBD induces detectable RBD-specific antibody responses.′

### Example 3 Repeated nRBD Treatment with cRBD Results in Strong Antibody Responses

Since the above antibody responses should be increased to ensure immune protection, we tested whether combining the 6 times repeated injection of nRBD with cRBD might boost the immune response. Therefore we pretreated the mice 6 times with nRBD prior to immunization cRBD (FIG. 3A).

WT mice obtained either repeated injections of native RBD (6 i.p. administrations of 50 ug each in absence of adjuvant, within 14 days; +PT), while control animals were pre-treated with PBS only (−PT). Subsequently all animals were immunized i. p. with 50 μg of native RBD complexed with streptavidin (cRBD) in presence of CpG-ODN #1826 and boosted 3 weeks later.

In fact, pretreatment of the mice with nRBD two weeks prior to immunizations resulted in greatly augmented immune response (FIG. 3B).

RBD-specific IgM and IgG was measured in both groups by ELISA and compared to titers measured in CI mice. Mice repeatedly exposed to native RBD prior to immunization with cRBD mount robust antibody responses against RBD, that can be detected already after the first application of cRBD.

Compared to cRBD without pretreatment (wo PT), pretreatment resulted in up to 40 fold higher concentration of anti-RBD IgM at d7 after cRBD immunization and this IgM response was further increased at d28, one week after secondary immunization (FIG. 3B). Anti-RBD IgG was also increased if nRBD pretreatment was combined with cRBD immunization as measured by the high titers of anti-RBD IgG at d14 and d28 (FIG. 3B). WT mice received either repeated injections of native RBD (6 i.p. administrations of 50 ug each in absence of adjuvant, within 14 days; +PT), while control animals were pre-treated with PBS only (−PT). Subsequently animals were immunized i. p. with 50 μg of native RBD complexed with streptavidin (cRBD, on day 0) in presence of CpG-ODN #1826 and boosted after 3 weeks and 5 weeks. A third group of mice was RBD-pre-treated but obtained primary immunization with cRBD 5 weeks later (FIG. 4A).

RBD-specific IgM and IgG was determined by ELISA:

96-well Maxisorp ELISA plates (Nunc) were coated over night with 50 μl/well of RBD at a concentration of 10 μg/ml.

After three washing steps with 200 μl ELISA washing buffer (PBS 0.1% Tween-20), unspecific binding sites were blocked for 1 h at 37° C. with 100 μl/well ELISA blocking buffer (PBS 1% BSA). After three additional washing steps with 200 μl/well ELISA washing buffer, 100 μl ELISA blocking buffer were added to each well. 150 μl of pre-diluted serum was applied in duplicates to the first row of the plate. By transferring 50 μl from the first row to the second and so on to the eighth row, serial dilutions at a ratio of 1:3 were prepared. Duplicate columns coated with either anti-mouse IgM (Southern Biotech, 1020-01) or IgG (Southern Biotech, 1030-01) at a concentration of 10 μg/ml, captured with mouse IgM (Southern Biotech, 0101-01) or IgG (Southern Biotech, 0107-01) served as standards. 2 wells containing only blocking buffer served as blank. For capturing, the plates were incubated for further 2 h at 37° C. Unbound antibodies were removed by washing three times with 200 μl/well ELISA washing buffer and 50 μl/well secondary goat a-mouse IgM (Southern Biotech 1020-04, diluted 1:1,000 in ELISA blocking buffer) or IgG antibody coupled with alkaline phosphatase (Southern Biotech 1030-04, diluted 1:2,000 in ELISA blocking buffer) solution were added and incubated for 1 h at 37° C. Afterwards, the plates were washed again three times with ELISA washing buffer to remove excess antibody. Substrate solution containing 4-nitrophenyl phosphate (pNPP, Gennaxon) in diethanolamine-buffer was added to each well. ODs were measured at 405 nm using a Multiskan FC ELISA plate reader (Thermo Fisher Scientific) and antibody concentrations were determined by using the Skanlt software provided with the machine.

Pre-treatment with native RBD primes for efficient antibody responses even if the primary immune response is delayed by 5 weeks.

Interestingly, the effect of pretreatment with nRBD seems to persist for extended period as immunization with cRBD at d35 after pretreatment induced robust antibody responses similar to those induced at do of immunization (FIG. 4).

These data show that pretreatment with nRBD strongly enhances the immune response induced by cRBD suggesting that repeated nRBD treatment may prime the immune system for efficient RBD-specific immune responses.

### Example 4 High Antibody Titer is Required for In Vitro Virus Neutralization

To test whether the amount of antibodies induced by the combined treatment was sufficient for virus neutralization, we performed in vitro neutralization assays using pseudo-virus preparations expressing the spike protein of Sars-COV 2 (Method is described in Hoffmann, M., et al., 2021, Cell, 184 (9), 2384-2393).

The data show that the serum of the pretreated mice showed evident capacity to prevent SARS-COV-2 infection in vitro (FIG. 5C). Moreover, the data also show that the weak immune responses induced by cRBD injection without pretreatment were not sufficient for virus neutralization (FIG. 5).

Moreover, we found that different ratios of sRBD to cRBD in the composition of the immunization cocktail result in different ratios of immunoglobulin isotypes (i.e. IgG to IgM) which allow refined control of immune responses after immunization.

Thus, combining nRBD treatment with cRBD immunization induces robust antibody responses for neutralizing Sars-COV 2 infection.

### Example 5 IgM BCR Expression Accelerates the Antibody Response

We immunized IgD-deficient mice in parallel to wildtype mice using the combined protocol of nRBD pretreatment and subsequent cRBD immunization. WT and IgD-KO mice were repeatedly exposed to native RBD (i.p. administrations of 50 μg each in absence of adjuvant, within 14 days; +PT). Subsequently all animals were immunized i. p. with 50 μg of native RBD complexed with streptavidin (cRBD) in presence of CpG-ODN #1826 and boosted 3 weeks later. Blood was collected from immunized mice at the indicated time points. RBD-specific IgM and IgG was measured in both groups by ELISA.

In agreement with the proposed role of IgD, primary (d7) and secondary (d28) IgM immune response was highly increased in IgD-deficient mice as compared with wild-type controls (FIG. 6). In contrast, secondary (d28) IgG antibody response was reduced in IgD-deficient mice (FIG. 6). This indicates that B cell populations, in which IgD expression is reduced or absent might elicit quicker immune responses after immunization with cRBD. This suggests that individuals with vital production of newly generated B cells, which have not yet reached the IgD-high stage, are well protected against viral infection because of quicker primary responses.

Responsiveness of B cells determines the strength and isotype of the antibody response. Newly generated B cells have a lot more IgM than IgD and are generated in the course of lymphopoiesis which declines with age. The difference between aged and young patients in surviving COVID-19 might be related to weak primary immune responses in the aged patients.

### Example 6 Robust Antibody Responses by RBD Complexes Generated by Chemical Crosslinking

The above experiments suggest that RBD complexes is important for eliciting immune responses and that native RBD is required for efficient priming of the immune response. However, the generation of immune complexes by biotinylating RBD and subsequent complex formation are unlikely to be practical for large-scale generation of vaccines. Therefore, we tested whether chemical crosslinking is capable of generating immunogenic cRBD. To this end, we used a chemical compound, 1,2-phenylene-bis-maleimide (thereafter referred to as bismale), that is typically used for irreversible crosslinking via sulfhydryl (SH) groups. We tested different concentrations and incubation times to generate different ratios of complex to native RBD (FIG. 7A). WT mice were immunized i. p. with 50 μg cRBD*MM in presence of CpG-ODN #1826. The immunization was boosted after 3 and 5 weeks with the same compounds (FIG. 7B).

After dialysis, we performed immunization experiments by injecting wildtype mice at do and d21 with similar amounts of chemically crosslinked RBD. The experiments show that moderate IgM amounts were detected at d28, one week after secondary immunization, while IgG was strongly increased at this time (FIG. 7C). Mice immunized with cRBD*MM which still contains monomeric RBD molecules, mount robust antibody responses with low RBD-specific IgM concentrations. These data show that chemical crosslinking produces mixtures of nRBD and cRBD that have an enormous capacity for induction of antigen-specific immune responses.

### Example 7 Antibodies Elicited by Chemically Crosslinked RBD Possess High Neutralization Capacity

The chemical crosslinking of RBD might provide a practical method for the production of SARS-COV 2 vaccines, as recombinant RBD can easily be produced and used for primary and secondary immunization in typical vaccination. Hence, we tested whether the resulting antibodies can prevent virus infection (Method is described in Hoffmann, M., et al., 2021, Cell, 184 (9), 2384-2393). The results show that mice immunized with the chemically crosslinked RBD possess a high capacity in neutralization assays using pseudo-virus preparations (FIG. 8).

These data suggest that chemical crosslinking of RBD allows the simple design of efficient vaccines against SARS-COV 2.

### Example 8 Activated Antigen Forms IgG Complexes that Boost Immune Responses

We noticed that chemical crosslinking with bismale slightly changed the behavior of monomeric RBD in Coomassie staining on SDS page (FIG. 7A). We analyzed the sequence of RBD and identified a single SH group which is not engaged in intramolecular disulfide bonds. We proposed that bismale treatment of RBD or other proteins may result in saturated binding of bismale so that no additional proteins can be crosslinked by a bismale molecule (FIG. 9B, middle). It is possible, however, that bismale treatment results in a monomeric RBD bound by bismale, in which a free maleimide group is still available (FIG. 9B, bottom).

RBD* was complexed with 20 μg bismale per 100 μg of RBD, while RBD** indicates complexation with 100 μg per 100 μg of RBD (FIG. 9C).

Immunization was performed in WT C57BL6/J mice using 50 μg of non-complexed native RBD (nRBD, n=3), 50 μg of RBD complexed with 10 μg bismale (RBD*, n=3) or 50 μg of RBD complexed with 10 μg bismale in the presence of 25 μg polyclonal murine IgG (RBD*IgG). 50 μg CpG-ODN #1826 was used as adjuvant in all conditions. IgM or IgA isotype was used instead of IgG for immunization with RBD*IgM and RBD*IgA. Mice were boosted with the identical immunization mixture 21 days after primary immunization. Serum was collected on day 28 for analysis. (FIG. 9D).

WT mice were immunized either with 50 μg of non-complexed native RBD+CpG-ODN (nRBD, n=3), 50 μg of RBD complexed with 10 μg bismale+CpG-ODN (RBD*, n=3) or 50 μg of RBD complexed with 10 μg bismale in the presence of 25 μg murine IgG but in absence of CpG-ODN (RBD*IgG, n=2).

This results in activated RBD that can undergo bioconjugation with other proteins in vitro or in vivo. Importantly, increasing amount of bismale results in a decrease of the monomeric RBD suggesting that more bismale leads to more protein complexes (FIG. 9C). To test the potential of forming heterocomplexes and at the same time to investigate the role of immunoglobulins in randomly formed complexes, we included IgM, IgA and IgG in the crosslinking reaction.

Interestingly, the results showed that, while IgM and IgA failed to boost the immune response, the crosslinking of RBD and IgG led to a dramatic increase of the RBD-specific immune response (FIG. 9D). Importantly, adding IgG after terminating the bismale mediated crosslinking did not boost the immune response suggesting that bismale mediated crosslinking is important for the IgG-mediated enhancement.

The enhancement observed by IgG prompted us to test whether IgG may act as adjuvant replacing conventional adjuvants such as alum or CpG. To this end, we compared the immune response generated by complex RBD injected in the presence of CpG or IgG as adjuvant. The results show that IgG containing immune complexes are capable of inducing robust antibody responses in the absence of conventional adjuvants such CpG or alum that activate TLRs. In conclusion, the data suggest that the generation of IgG containing immune complexes by crosslinking IgG and a particular antigen in vitro, or in vivo by injecting the antigen after incubation with bifunctional crosslinkers containing two reactive groups in vitro. Such activated antigens represent a simple and efficient way for the development and production of effective vaccines.

### Example 9

Treating the bismaleimide-crosslinked immune complexes with cysteine in vitro results in quenching of still available reactive maleimide groups and reversion of antigen activation thereby reducing antibody production (FIG. 10D).

100 μg activated RBD were quenched in at least 1 μl of freshly prepared 2 M L-Cysteine (Sigma-L-Cysteine BioUltra, ≥98.5% 30089-25G) solution and incubated over night at RT. The following day the sample was rebuffered at 4° C. under constant agitation with a magnetic stir bar by using a dialysis cassette (Thermo Fisher Scientific Slide-A-Lyzer™10K MWCO 66381) to remove unbound cysteine and maleimide. 1×PBS was changed after over night dialysis and the sample was dialysed for further 4 to 6 hours at 4° C. under constant agitation with a magnetic stir bar.

The data show that increased maleimide (RBD**) results in increased antibody responses and that quenching the maleimide-treated antigen with cysteine (RBD**C) reduces the antibody responses dramatically. This suggests that maleimide treatment led to the generation of activated antigen, which is capable of generating complexes in vivo and this capacity is important for the immune response.

Thus activating the antigen, by making it reactive with SH groups on autoantigens, amplifies the immune response. Including total IgG in the antigen activation leads to the generation of protein complexes that mimic immune complexes thereby inducing efficient antibody responses.

### Example 10

Antigen (Ag) complexes were generated by biotinylation and subsequent incubation with streptavidin (SAV). The complex antigen induces antibody responses. Multivalency depends on the number of biotins per molecule. Multiple biotin groups allow multiple SAV binding and higher molecular complexes. Crosslinking with SAV leads to higher molecular complexes and efficient immune responses (FIG. 11, 12).

1-25. (canceled)

26. A method for treating/preventing an infection, said method comprises administering to a subject a therapeutically effective amount of a composition comprising:
(i) a mono/divalent antigen particle, comprising an antigenic portion comprising one or two antigenic structures capable of inducing an antibody-mediated immune response against a target antigen, wherein the target antigen is a pathogen-associated antigen, and
(ii) a polyvalent antigen particle comprising an antigenic portion comprising more than two antigenic structures capable of inducing an antibody-mediated immune response against the target antigen and wherein the more than two antigenic structures are cross-linked, wherein the target antigen is a pathogen-associated antigen.

27. The method of claim 26, wherein the more than two antigenic structures comprise multiple identical antigenic structures.

28. The method of claim 26, wherein the polyvalent antigen particle further comprises a carrier portion which is coupled to the antigenic portion and/or wherein the mono/divalent antigen particle further comprises a carrier portion which is coupled to the antigenic portion.

29. The method of claim 28, wherein the carrier portion comprises a structure selected from the group consisting of polypeptides, immune CpG islands, limpet hemocyanin (KLH), tetanus toxoid (TT), cholera toxin subunit B (CTB), bacteria or bacterial ghosts, liposome, chitosome, virosomes, microspheres, dendritic cells, particles, microparticles, nanoparticles, and beads.

30. The method of claim 26, wherein the cross-link in the polyvalent antigen particle is a chemical cross-link, such as a bis-maleimide mediated cross-link, or is a protein cross-link, such as a biotin-streptavidin mediated cross-link.

31. The method of claim 26, wherein the polyvalent antigen particle comprises a complex of the following formula A-L-A, wherein A is a target antigen comprising portion, and wherein L is a linker of the cross link; optionally wherein L is a bismaleimide; or optionally wherein the complex is of the following structure (I), wherein R is a target antigen comprising portion:

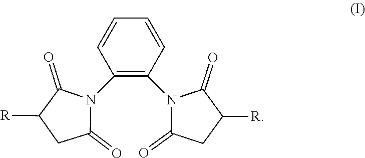

32. The method of claim 26, wherein the polyvalent antigen particle comprises a linker with a crosslink reactive group for protein conjugation, optionally a linker with a crosslink reactive group for stable protein conjugation.

33. The method of claim 32, wherein the crosslink reactive group is a group selected from carboxyl-to-amine reactive groups, amine-reactive groups, sulfhydryl-reactive groups, aldehyde-reactive groups and photoreactive groups, or wherein the crosslink reactive group is a group selected from carbodiimide, NHS ester, imidoester, pentafluorophenyl ester, hydroxymethyl phosphine, maleimide, haloacetyl, hydrazide, alkoxyamine, diazirine and aryl azide.

34. The method of claim 26, wherein the polyvalent antigen particle comprises the at least two copies of the antigenic structure in spatial proximity to each other, optionally within a range of 3 nm to 20 nm.

35. The method of claim 26, wherein the polyvalent antigen particle is linked to an adjuvant, wherein the adjuvant is IgG.

36. The method of claim 26, wherein treatment and/or prevention comprises at least two administration time points.

37. The method of claim 36, wherein prevention comprises administering the mono/divalent antigen particle before the polyvalent antigen particle.

38. The method of claim 36, wherein the treatment and/or prevention comprises at least two administration time points for the mono/divalent antigen particle and least two administration time points for the polyvalent antigen particle.

39. The method of claim 26, wherein the antibody-mediated immune response is an IgG and/or IgM mediated immune response.

40. The method of claim 26, wherein the infection is a viral infection.

41. The method of claim 40, wherein the viral infection is a coronavirus infection.

42. The method of claim 41, wherein the coronavirus infection is a SARS-COV-2 infection.

43. The method of claim 42, wherein the pathogen-associated antigen comprises an amino acid sequence derived from a corona virus spike protein, optionally wherein the amino acid sequence is derived from a receptor binding domain (RBD) sequence, optionally a complete RBD sequence, or a sequence comprising at least 80% sequence identity to the amino acid sequence of the SARS-COV-2 RBD amino acid sequence (SEQ ID NO: 1).

44. A method for producing an antibody that binds to a pathogen-associated antigen comprising the steps of:
(1) administration of:
(i) a mono/divalent particle, comprising an antigenic portion comprising one or two antigenic structures capable of inducing an antibody-mediated immune response against a target antigen, wherein the target antigen comprises a pathogen-associated antigen, and
(ii) a polyvalent antigen particle comprising an antigenic portion comprising more than two antigenic structures capable of inducing an antibody-mediated immune response against the target antigen and wherein the more than two antigenic structures are cross-linked, wherein the target antigen comprises a pathogen-associated antigen,
to a subject and/or a cell capable of producing antibodies; and
(2) isolating an antibody from the subject and/or cell, wherein the antibody binds to the target antigen.

45. A method for treating/preventing an infection, said method comprises administering to a subject a therapeutically effective amount of polynucleotide encoding:
(i) a mono/divalent antigen particle, comprising an antigenic portion comprising one or two antigenic structures capable of inducing an antibody-mediated immune response against a target antigen, wherein the target antigen comprises a pathogen-associated antigen, and
(ii) a polyvalent antigen particle comprising an antigenic portion comprising more than two antigenic structures capable of inducing an antibody-mediated immune response against the target antigen and wherein the more than two antigenic structures are cross-linked, wherein the target antigen comprises a pathogen-associated antigen.

46. A method for treating/preventing an infection, said method comprises administering to a subject a therapeutically effective amount of a vector comprising the polynucleotide of claim 45.
